(19) United States
(12) Patent Application Publication
(10) Pub. No.: US 2011/0110820 A1
(43) Pub. Date: May 12, 2011

(54) METHOD AND ENCLOSURE FOR SANITIZING HARD AND SOFT GOODS

(76) Inventor: Walter B. MANN, Johns Creek, GA (US)

(21) Appl. No.: 12/853,862

(22) Filed: Aug. 10, 2010

Related U.S. Application Data
(60) Provisional application No. 61/232,874, filed on Aug. 11, 2009.

(51) Int. Cl. A61L 2/20

(52) U.S. Cl. 422/30; 422/28; 422/105

(57) ABSTRACT

A method of sanitizing hard and soft goods comprising placing the goods within an a sealable enclosure; sealing the enclosure; producing ozone within the sealed enclosure; circulating the ozone within the sealed enclosure to sanitize the goods; adjusting the environment within the enclosure to optimize the sanitizing properties of and reduce the potential for the ozone to leak to the surrounding environment; removing the ozone to a prescribed safe concentration level; and maintaining the seal of the enclosure until the ozone concentration reaches the prescribed safe concentration level. The enclosure includes an open side; a sealing door for sealing the sealable enclosure; a locking mechanism; a circulation device, a fresh air heater; a vacuum pump, an ozone generator; an ozone destructor, wherein the ozone generator and ozone destructor are disposed within the sealable enclosure; and a humidification device to adjust the relative humidity inside the sealed enclosure.

This application claims the benefit of U.S. Provisional Patent Application No. 61/232,874, filed on Aug. 11, 2009, which is hereby incorporated by reference for all purposes as if fully set forth herein.

### BACKGROUND OF THE INVENTION

1. Field of the Invention

The present invention relates to a method and an enclosure for treating hard and soft goods to sanitize and remove odor from the same by using ozone.

2. Background Art

The problem of bacteria, viruses and mold growth on hard and soft goods is common. These hard and soft goods are commonly used in areas such as sports, public safety, military, daycare, hospitality, theater, band, and other communal environments. In some instances, the hard and soft goods may be used over and over again, by many people. These people may have communicable illnesses or have hands laden with infectious bacteria and/or viruses that can be spread to others. These situations can be particularly problematic when there are contagious diseases or disease outbreak.

To prevent the spread of mold or diseases from bacteria and viruses, the hard and soft goods should be sanitized. However, while clothing can be washed and dried in a convenient manner such as laundering in a washing machine, many hard and soft goods cannot be cleaned or sanitized in a similar method. For example, in the sports of lacrosse and football players wear bulky protective padding and hard helmets, which cannot be put in a washing machine and/or dryer. In addition, protective equipment worn by military and law enforcement personnel cannot be similarly washed. For example, body armor worn by soldiers in the field cannot be easily washed. Further, many of the common implements used in daycare centers and hotels cannot be similarly sanitized.

It is known in the art that ozone can be used to sanitize such goods. For example, U.S. Pat. No. 6,889,449 discloses a method for sanitizing sports equipment using ozone. Similarly, U.S. Pat. No. 6,134,806 discloses a sports equipment bag with an air distributor and an ozone generator for drying and removing bacteria and fungus from clothing and athletic equipment.

However, none of the prior art deals with the need to provide a completely sealed enclosure while managing the humidity, temperature, pressure and ozone level inside an enclosure to maximize the sanitizing properties of the ozone. Furthermore, once sanitization is complete, the ozone must be destructed to levels prescribed as safe by various government regulations before the device is allowed to be opened and the residual gas is vented to the surrounding environment.

While the above patents do teach various methods and devices to sanitize goods using ozone, there still exists a need for a method and apparatus for efficient sanitization in an environmentally responsible manner.

### BRIEF SUMMARY OF THE INVENTION

Accordingly, the present invention is directed to methods and enclosure for sanitizing hard and soft goods that substantially obviates one or more of the problems due to limitations and disadvantages of the related art. Embodiments of the present invention provide a method and an apparatus for sanitizing hard and soft goods utilizing a sealable enclosure with ozone.

One embodiment is directed to a method of sanitizing hard and soft goods comprising the steps of: placing the hard and soft goods within an a sealable enclosure; sealing the enclosure; producing ozone containing air within the sealed enclosure; circulating the ozone containing air within the sealed enclosure to sanitize the hard and soft goods; adjusting the environment within the enclosure to ensure that the sanitizing properties of the ozone are optimized and the potential for leakage to the surrounding environment is reduced; removing the ozone to a prescribed safe concentration level; and maintaining the seal of the enclosure until the ozone concentration reaches a prescribed safe concentration level.

Another embodiment is directed to an enclosure for sanitizing hard and soft goods comprising: a sealable enclosure with a sealing door for sealing the sealable enclosure, a locking mechanism, a fresh air heater; and a vacuum pump; an ozone generator; an ozone destructor, wherein the ozone generator and ozone destructor are disposed within the sealable enclosure. The enclosure further comprises a humidification device to adjust the relative humidity inside the sealed enclosure. In some embodiments, the enclosure can also have a dehumidification device disposed within the enclosure. In further embodiments, the enclosure can also have a cooling device disposed within or external to the enclosure to reduce the operating temperature inside the enclosure. In addition, an ozone detector can be disposed within the enclosure or on an exterior surface of the enclosure.

Further embodiments, features, and advantages of the method and enclosure for sanitizing hard and soft goods, as well as the structure and operation of the various embodiments of the method and enclosure for sanitizing hard and soft goods, are described in detail below with reference to the accompanying drawings.

It is to be understood that both the foregoing general description and the following detailed description are exemplary and explanatory only, and are not restrictive of the invention as claimed.

### BRIEF DESCRIPTION OF THE DRAWINGS/FIGURES

The accompanying figures, which are incorporated herein and form part of the specification, illustrate a method and an enclosure for sanitizing hard and soft goods. Together with the description, the figures further serve to explain the principles of the method and the enclosure for sanitizing hard and soft goods described herein and thereby enable a person skilled in the pertinent art to make and use the method and the enclosure for sanitizing hard and soft goods.

FIG. 1 is a flowchart illustrating a method of sanitizing hard and soft goods.

FIG. 2 is a perspective view of an embodiment of a sealable enclosure for sanitizing hard and soft goods.

FIG. 3 is a front view of an embodiment of a sealable enclosure for sanitizing hard and soft goods.

FIG. 4 is a side view of an embodiment of a sealable enclosure for sanitizing hard and soft goods.

### DETAILED DESCRIPTION OF THE INVENTION

Reference will now be made in detail to embodiments of the method and apparatus for sanitizing hard and soft goods using ozone with reference to the accompanying figures, in which like reference numerals indicate like elements.

Embodiments of the method and the enclosure relate to sanitizing hard and soft goods by utilizing ozone in a sealable enclosure.

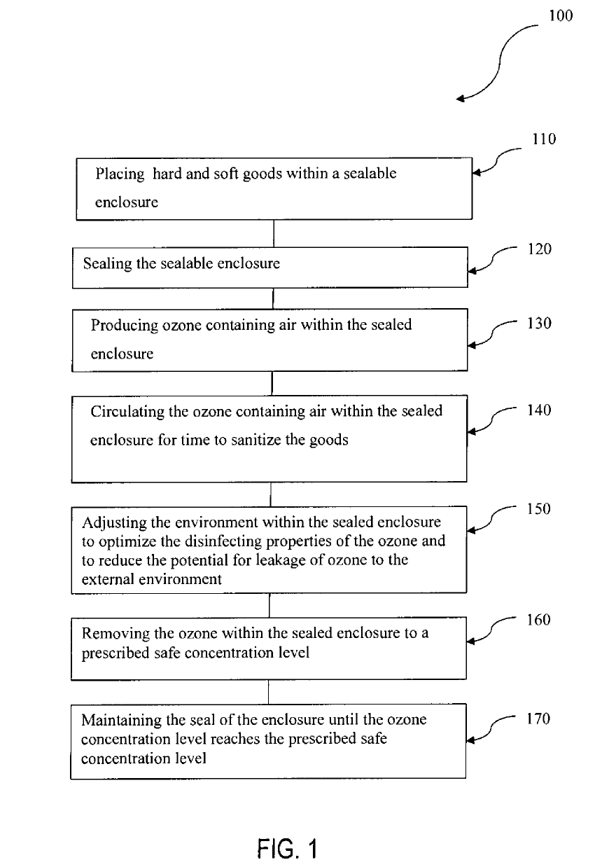

As shown in FIG. 1, an embodiment is direct to a method for sanitizing hard and soft goods 100 including the steps of placing the hard and soft goods within an a sealable enclosure 110; sealing the enclosure 120; producing ozone containing air within the sealed enclosure 130; circulating the ozone containing air within the sealed enclosure to sanitize the hard and soft goods 140; adjusting the environment within the enclosure to optimize the sanitizing properties of the ozone and reduce the potential for ozone to escape and leak to the surrounding environment 150; removing the ozone to a prescribed safe concentration level 160; and maintaining the seal of the enclosure until the ozone concentration reaches a prescribed safe concentration level 170.

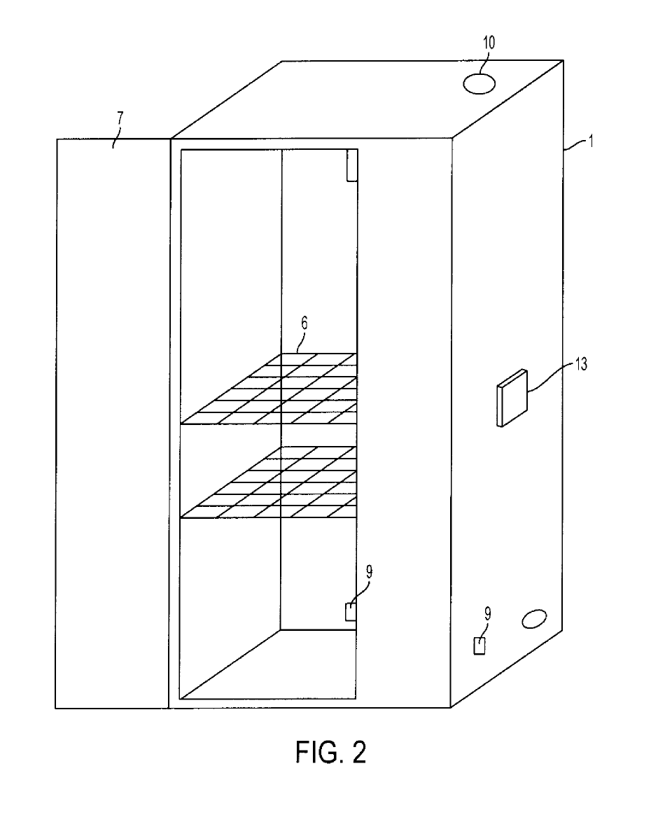

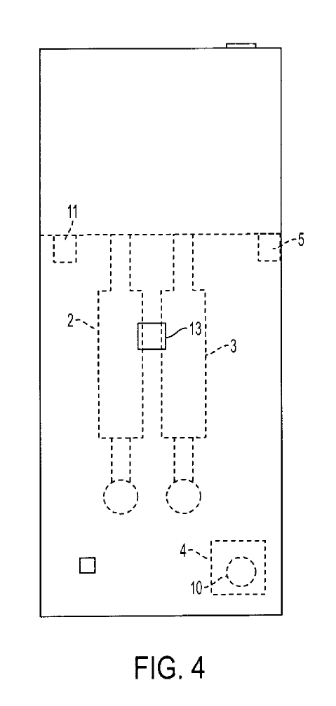

In step 110, hard and soft goods can be placed within an interior space in a sealable enclosure 1 for sanitizing the goods using ozone. As illustrated in FIGS. 2-4, sealable enclosure 1 is a three-dimensional structure with one open side for accessing the interior space of the sealable enclosure. In some embodiments, the sealable enclosure 1 can include shelving 6 on which the hard and soft goods can be placed. In further embodiments, the sealable enclosure can include hooks on which the hard and soft goods can be hung.

After the goods are placed within the sealable enclosure 1, in step 120 the enclosure can be sealed. In one embodiment, the sealable enclosure 1 is provided with an access door 7 attached to the open side. The access door 7 can be closed to seal the open side of the sealable enclosure. In another embodiment, the sealable enclosure 1 can be an isolation tent or shelter that can be sealed with a zipper or the like. It is important that the sealable enclosure 1 is sealed to reduce the potential of the ozone produced within the enclosure from escaping and leaking to the external environment. As such, in some embodiments, the sealable enclosure 1 can be further provided with a locking mechanism 8 to ensure that the access door 7 creates an airtight seal with the sealable enclosure 1.

In step 130, ozone containing air can be produced within the sealed enclosure. In particular, an ozone generator 2 can be used to introduce ozone within the sealed enclosure to produce ozone containing air within the enclosure. The generated ozone can be used to sanitize the hard and soft goods by destroying any bacteria, viruses, and mold that can be lingering on the hard and soft goods.

In one embodiment the ozone generator 2 may be external to the sealed enclosure. Accordingly, the ozone generator 2 is fluidly connected to the sealed enclosure through a fluid passageway so that the ozone can flow into the interior space of the sealed enclosure. In other embodiments, the ozone generator 2 can be disposed within the interior of the enclosure 1. By locating the ozone generator 2 wholly within the interior of the enclosure, the need for a fluid passageway is reduced. For example, the ozone generator 2 disposed within the enclosure 1 can be directly vented into the enclosure thereby reducing the potential for leakage of ozone to the exterior environment.

As illustrated in FIG. 1, the method further includes step 140. In step 140, the produced ozone containing air is circulated within the sealed enclosure. The ozone containing air can be circulated throughout the sealed enclosure to assure that it contacts all of the hard and soft goods placed within. The enclosure can be provided with a circulation device such as a fan, a blower or other device suitable for circulating the ozone containing air within the sealed enclosure. For example, in one embodiment, any conventional off-the-shelf fan can be disposed within the enclosure and activated to circulate the ozone containing air. In another embodiment, a blower can be disposed within the enclosure to disperse the ozone containing air throughout the enclosure.

Further, the ozone containing air is circulated for a time sufficient to sanitize the hard and soft goods. Sanitizing the goods refers to killing approximately 99.9% of the bacteria, viruses, mold or other microorganism on the surfaces of the goods. As the ozone concentration level is increased, the time sufficient for sanitizing the goods decreases. In some embodiments if the ozone concentration is between about 10 to about 30 parts per million (ppm), the ozone containing air can be circulated for a time ranging from about 5 minutes to about 60 minutes. In other embodiments, the ozone concentration can be reduced and the ozone containing air can be circulated for longer periods of time, such as, 6 hours, 8 hours or 12 hours. For example, in one embodiment, a user at the end of a day can place the hard or soft goods (e.g. toys at a daycare center) to be sanitized inside the enclosure and the ozone containing air can be circulated overnight so that goods are sanitized by the next morning and ready for another day of shared usage.

To further optimize the sanitizing properties of the ozone, in step 150, the environment within the sealed enclosure can be adjusted. For example, the relative humidity, temperature, or ozone concentration can be changed within the sealed enclosure.

In one embodiment, the step of adjusting the environment in the sealed enclosure can comprise changing the relative humidity inside the sealed enclosure. Because the effectiveness of ozone as a sanitizing agent can be enhanced at higher relative humidity, the relative humidity inside the sealed enclosure can be increased. For example, the relative humidity inside the sealed enclosure can be adjusted to be greater than 40% for at least a portion of the time the ozone containing air is circulated to sanitize the hard and soft goods. In another embodiment, the relative humidity inside the sealed enclosure can be greater than 60% for at least a portion of the time the ozone containing air is circulated for sanitizing the hard and soft goods. Further, the relative humidity inside the sealed enclosure can be greater than 80% for at least a portion of the time the ozone containing air is circulated to sanitize the hard and soft goods.

To change the relative humidity inside the sealed enclosure 1, the sealed enclosure 1 can be further provided with a humidification device 5 to increase the relative humidity. In further embodiments, the sealed enclosure 1 can be provided with a dehumidification device 11 to decrease the relative humid in the enclosure.

In addition to adjusting the relative humidity, the temperature inside the sealed enclosure can be adjusted to maximize the effectiveness of the ozone in sanitizing the goods. In some embodiments, the temperature inside the enclosure during operation can be the same or similar to the surrounding ambient temperature. The temperature inside the enclosure can range from 32° F. to over 115° F.

In other embodiments, the temperature inside the enclosure can be increased or decreased in comparison to the surrounding ambient temperature. For example, if the temperature inside the enclosure is below freezing at 32° F., warm air can be injected into the interior of the enclosure via a heater to raise the temperature inside the enclosure. Alternatively, when the ambient temperature surrounding the enclosure is over 100° F., a cooling unit can be used to decrease the temperature inside the enclosure because it can be easier to generate ozone at a lower temperature.

Aside from adjusting the relative humidity and temperature, the ozone concentration level inside the enclosure can be adjusted to increase the efficiency of sanitizing the hard and soft goods in the enclosure. Because it requires more time to sanitize the hard and soft goods when the ozone concentration is low, the ozone concentration level can be increased to reduce the time required for sanitizing the goods. However, too high of an ozone concentration level can cause destruction to the goods (e.g. ozone concentration levels in excess of 50 ppm damage the surfaces of certain hard or soft goods). For example, in some embodiments, the ozone concentration in the enclosure can range from 10 ppm to 45 ppm.

In further embodiments, adjusting the environment within the sealed enclosure can include adjusting the pressure within the sealed enclosure. Adjusting the pressure within the sealed enclosure has the added benefit of minimizing the potential for the ozone containing air from escaping due to a pressure differential. For example, in one embodiment, the potential for the ozone to escape and leak from the sealed enclosure is minimize by reducing the pressure inside the sealed enclosure to less than 1 atmosphere.

As shown in FIG. 1, step 160 comprises removing the ozone within the sealed enclosure to a prescribed safe concentration level. Because most countries have regulations mandating that ozone cannot be created and vented to the atmosphere, the ozone must be removed from the within the sealed enclosure to a prescribed safe concentration level. In one embodiment, the prescribed safe concentration level is equal to or less than 1 part per million (ppm) in the enclosure. In other embodiments, the prescribed safe level can be 0.3 ppm or 0.1 ppm inside the enclosure. Further, the seal of the enclosure is maintained until the ozone has been removed such that the ozone concentration level reaches the prescribed same concentration level.

To remove the ozone to the prescribed safe concentration level, the ozone can be thermally or catalytically destroyed to be reduced to oxygen. In one embodiment, a catalyst can be used to remove the ozone to the prescribed safe concentration level. For example, a metal oxide (e.g. manganese oxide, ceric oxide, or chromic oxide), activated carbon or any other known catalyst that is suitable for destroying ozone can be used.

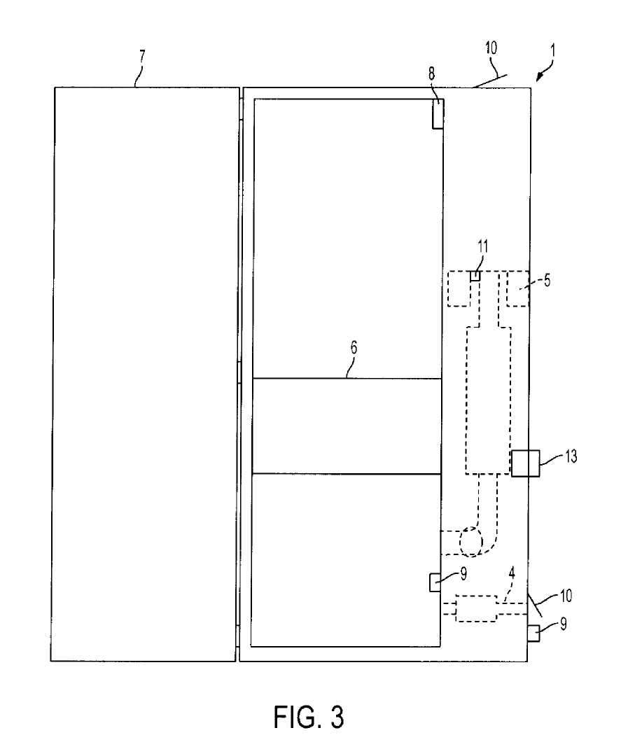

To destroy the ozone, any conventional off-the shelf ozone destructor 3 can be adapted and used to remove the ozone within the enclosure. For example, in one embodiment, the ozone destructor can be exterior to the enclosure 1. In other embodiments, the ozone destructor 3 can be disposed within the enclosure 1, as shown in FIG. 3.

After the ozone has been removed to a prescribed safe concentration level, the sealed enclosure can be opened and the sanitized hard and soft goods can be removed.

In some embodiments, the method of sanitizing hard and soft goods can further comprise drying the hard and soft goods after the ozone is removed. For example, the goods can be dried by introducing fresh warm air into the enclosure. To introduce fresh warm air, the sealable enclosure can further include a fresh air heater 4. In some other embodiments, the goods can be dried by circulating dry air (i.e. air with low humidity) in the enclosure.

Another embodiment is directed to an enclosure for sanitizing hard and soft goods using ozone. As shown in FIGS. 2-4 an enclosure for sanitizing hard and soft goods comprises a sealable enclosure 1; an ozone generator 2; and an ozone destructor 3. In some embodiments, the enclosure for sanitizing hard and soft goods can further include a humidification device 5. In even further embodiments, the enclosure for sanitizing hard and soft goods can additionally include a dehumidification device 12. The enclosure can further include a heater 4, a vacuum pump or blower 22 and an optional cooling device 13.

As illustrated in FIGS. 2-4, the sealable enclosure 1 includes a sealing/access door 7; a locking mechanism 8; sealing vents 10; a fresh air heater 4 and a vacuum pump 11. The sealable enclosure 1 can also be provided with shelves 6 on which the hard and soft goods for sanitizing can be place. In some embodiments, the sealable enclosure 1 may further comprise hooks (not shown) from which goods can be hung.

The sealable enclosure 1 can be any three dimensional structure known to one of ordinary skill in the art that is suitable for housing hard and soft goods for the amount of time sufficient for sanitizing the goods. The sealable enclosure 1 can be a rigid or soft enclosure. Furthermore, the sealable enclosure 1 can be any size suitable for housing a plurality of hard and soft goods. For example, in one embodiment, the sealable enclosure 1 can be a cabinet. In other embodiments, the sealable enclosure can be as large as a commercial trailer or a dedicated room for sanitizing hard and soft goods in large quantities at one time or goods of larger sizes. In further embodiments, the sealable enclosure can be a sealable isolation tent or shelter, such as the ISO-POD™ by Immediate Response Technologies.

The sealable enclosure 1 includes one open side as an entry/access port for placing the hard and soft goods within the sealable enclosure 1. The sealing door 7 is hingedly attached to the open side for closing the sealable enclosure 1. When sealing door 7 is closed, the locking mechanism 8 can be engaged to ensure that the enclosure 1 is securely sealed.

In some embodiments, the locking mechanism 8 can be affixed to the sealing door 7. For example, the locking mechanism 8 can be a locking handle disposed on the sealing door 7. In other embodiments, the locking mechanism 8 can also be fixed to the enclosure 1. For example, the locking mechanism can be an electronic lock, a magnetic lock, or any other locking mechanisms suitable for securing the seal between the sealing door 7 and enclosure 1 known to one of ordinary skill in the art.

In other embodiments, when the sealable enclosure is a soft-sided structure, such as an isolation tent, the sealing door 7 can be a flap or a plurality of flaps of fabric sown to the sides of the enclosure. The flap or flaps can be closed with a zipper or the like to seal the enclosure.

The sealable enclosure 1 further can optionally include a plurality of sealing vents 10 so that fresh air can be introduced into the enclosure to aid in drying of the hard and soft goods. Sealing vents 10 can be disposed on a plurality of surfaces of the enclosure 1. For example, sealing vents 10 can be disposed on a top surface, bottom surface, and/or side surface of the sealable enclosure 1. The sealing vents 10 are closed during operation of the enclosure to prevent ozone from escaping to the surrounding environment. After the ozone has been destructed to a safe level, the sealing vents 10 can be opened so that fresh air can enter the enclosure from the surrounding environment. In alternative embodiments, a fresh air heater 4 can be fluidly contained to a sealing vent 10 so that warm air can be introduced into the enclosure to aid in drying the hard and soft goods after sanitizing.

Additionally, the sealable enclosure 1 can include the fresh air heater 4 and a vacuum pump or blower 11.

The fresh air heater 4 can be used to dry the hard and soft goods in the enclosure after they have been sanitized with ozone. For example, the fresh air heater 4 can be used to introduce fresh warm air into the enclosure after the ozone concentration has diminished inside the enclosure. The fresh warm air can be circulated inside the sealed enclosure to dry the sanitized goods. The air heater 4 can be used to introduce warm air into the enclosure and raise the temperature inside the enclosure.

The vacuum pump or blower 11 is provided to adjust the pressure inside the sealable enclosure 1. The vacuum pump or blower can be provided as a safety device to prevent the ozone from leaking out of the enclosure. In particular, adjusting the pressure within the sealed enclosure has the benefit of minimizing the potential for the ozone containing air inside the sealed enclosure from escaping due to a pressure differential. For example, in one embodiment, the potential for the ozone to escape and leak from the sealed enclosure is minimize by reducing the pressure inside the sealed enclosure to less than 1 atmosphere. Accordingly, the vacuum pump or blower 11 can be used to reduce the pressure inside the sealable enclosure.

The vacuum pump 11 can be any conventional off-the-shelf vacuum pump or blower. For example, the vacuum pump 11 can be a positive displacement pump, a momentum transfer pump, an entrapment pump or any other type of vacuum pump known to one of ordinary skill in the art that can reduce the pressure inside the sealed enclosure.

Further, the sealable enclosure 1 is fluidly connected to an ozone generator 2. The ozone generator 2 can be used to produce and introduce ozone into the sealed enclosure. The ozone generator 2 can be any conventional ozone producing module. For example, in one embodiment, the ozone generator 2 can be a corona discharge module that produces ozone by passing air through a corona discharge between two parallel or concentric electrodes separated by a dielectric to break down the oxygen into charged oxygen atoms that can recombine to form molecules of ozone. In other embodiments, the ozone generator 2 can be a ultra-violet (UV) ozone generator, or a vacuum-ultraviolet (VUV) ozone generator that employ a light source that generates a narrow-band ultraviolet light to generator ozone. In further embodiments, the ozone generator can be a cold plasma module in which pure oxygen gas is exposed to plasma created by dielectric barrier discharge. The diatomic oxygen is split into single atoms, which then recombine in triplets to form ozone.

In one embodiment the ozone generator 2 may be external to the sealed enclosure. Accordingly, the ozone generator 2 is fluidly connected to the sealed enclosure through a fluid passageway so that the ozone can flow into the interior space of the sealed enclosure. In other embodiments, the ozone generator 2 can be disposed within the interior of the enclosure 1. By locating the ozone generator 2 wholly within the interior of the enclosure, the need for a fluid passageway is reduced and the potential for the ozone to escape to the surrounding environment is reduced. For example, the ozone generator 2 disposed within the enclosure 1 can be directly vented into the enclosure thereby decreasing the potential for leakage of ozone to the exterior environment.

The ozone containing air can be circulated throughout the sealed enclosure to assure that it contacts all of the hard and soft goods placed within to sanitize the goods. The enclosure can be provided with a circulation device such as a fan, a blower or other device suitable for circulating the ozone containing air within the sealed enclosure. For example, in one embodiment, any conventional off-the-shelf fan can be disposed within the enclosure and activated to circulate the ozone containing air. In another embodiment, a blower can be disposed within the enclosure to disperse the ozone containing air throughout the enclosure

To further enhance or optimize the sanitizing properties of the ozone, the environment within the sealed enclosure can be manipulated. For example, the relative humidity or temperature can be changed within the sealed enclosure.

In one embodiment, the sealed enclosure 1 can be provided with a humidification device 5 to increase the relative humidity. The humidification device 5 can be disposed within the interior space of the enclosure 1. The humidification device 5 can be any conventional off-the-shelf device known to one of ordinary skill in the art. In other embodiments, the humidification device 5 can be external and fluidly connected to the interior space of the enclosure 1. For example, the water reservoir of the humidification device can be external to the enclosure 1 to allow for easy access to filling or empty the reservoir. In such embodiments, the water reservoir can fluidly communicate with the enclosure 1 through a fluid passageway or conduit. For example, the humidification device can be a sprayer with a nozzle that can introduce a mist into the enclosure 1 to increase the relatively humidity of the internal environment.

The humidification device 5 can be used to adjust the relative humidity inside the sealed enclosure to be greater than 40% for at least a portion of the time to sanitize the hard and soft goods. In another embodiment, the relative humidity inside the sealed enclosure can be greater than 60% for at least a portion of the time for sanitizing the hard and soft goods. Further, the relative humidity inside the sealed enclosure can be greater than 80% for at least a portion of the time to sanitize the hard and soft goods.

In further embodiments, the sealed enclosure 1 can be optionally provided with a dehumidification device 12 to decrease the relative humidity in the enclosure 1. The dehumidification device 12 can also be disposed within the interior space of enclosure 1.

In addition to adjusting the relative humidity, the temperature inside the sealed enclosure can be adjusted to maximize the effectiveness of the ozone in sanitizing the goods. To adjust the temperature, the air heater 4 can be used to increase the temperature inside the enclosure 1. For example, if the temperature inside the enclosure is below freezing at 32° F., warm air can be injected into the interior of the enclosure via the heater 4 to raise the temperature inside the enclosure.

In some embodiments, the enclosure can be provided with a cooling unit 13. The cooling unit 13 can be used in addition to the heater 4 or as an alternative to heater 4. In one exemplary embodiment, the cooling unit can be 13 disposed within the interior of the enclosure 1. In other embodiments, the cooling unit 13 can be external and fluidly communicate with the enclosure 1 to introduce cooler air into the interior space of the enclosure 1.

For example, when the ambient temperature surrounding the enclosure is over 100° F., the cooling unit 13 can be used to decrease the temperature inside the enclosure because it can be easier to generate ozone at a lower temperature. The cooling unit 13 can be any conventional off-the-shelf cooling unit such as a condensing type, a compressor type or any other type of cooling device known to one of ordinary skill in the art that can be readily adapted to fluidly communicate with the interior environment of the enclosure.

Additionally, because most countries have regulations mandating that ozone cannot be created and vented to the atmosphere, the ozone created by the ozone generator 2 must be removed from within the sealed enclosure to a prescribed safe concentration level. Accordingly, the enclosure 1 includes an ozone destructor 3.

To destroy the ozone, any conventional off-the shelf ozone destructor 3 can be adapted and used to remove the ozone within the enclosure. For example, in one embodiment, the ozone destructor can be exterior to the enclosure 1. In other embodiments, the ozone destructor 3 can be disposed within the enclosure 1, as shown in FIG. 3.

To remove the ozone to reach a prescribed safe concentration level, the ozone destructor 3 can thermally or catalytically destroy the ozone to reduce it to oxygen. In one embodiment, a catalyst can further be used to remove the ozone to the prescribed safe concentration level. For example, a metal oxide (e.g. manganese oxide, ceric oxide, or chromic oxide), activated carbon or any other known catalyst that is suitable for destroying ozone.

To further ensure that the prescribed safe ozone concentration level is reached an ozone detector 9 can be disposed within the interior space of the enclosure 1. The ozone detector 9 can be any convention off-the-shelf ozone sensor, detector, monitor or analyzer that can determine the ozone concentration level within the sealed enclosure.

Additionally, to ensure that ozone is not escaping the enclosure and leaking to the surrounding environment, a second ozone detector can be optionally disposed outside of the enclosure. For example, an external ozone detector can be affixed to an external surface of the enclosure. In other embodiments, the external ozone detector can be located in a vicinity near or an area surrounding the enclosure that is within detector's range.

In operation, the enclosure 1 can be used to sanitize hard and soft goods. Hard and soft goods can be placed within the sealable enclosure 1 to kill any bacteria, viruses, mold, mildew or odors. The goods can be placed on shelves 6 in an arrangement that takes advantage of the fact that ozone is denser than air. Alternatively, the goods can be hung from hooks disposed within the interior space of the enclosure. After the goods for sanitizing have been place inside the enclosure, the enclosure 1 can be sealed shut by closing the sealing door 7 and engaging the locking mechanism 8.

Once the enclosure is sealed, ozone containing air can be produced within the sealed enclosure by the ozone generator 3. If the ozone generator 3 is disposed within the enclosure, it can directly vent the ozone into the enclosure. In embodiments where the ozone generator is disposed outside of the enclosure, the produced ozone can flow from the generator 3 through a fluid passageway into enclosure 1.

After ozone is generated, the ozone can be circulated within the sealed enclosure for a time to sanitize the hard and soft goods. The ozone should be circulated to contact all the goods within the sealed enclosure 1. In some embodiments, the ozone containing air can be circulated for 5 to 35 minutes. For example, in one exemplary embodiment, the total operation time of the enclosure can be 30 minutes and the ozone containing air can be circulated for 22 minutes while the ozone destruction phase takes about 8 minutes. In another exemplary embodiment, the total operation time can be about 16 minutes and the ozone circulation phase can last 12 minutes while the ozone destruction phase is about 4 minutes in duration. In further embodiments, the ozone circulation phase can be 60 minutes, 6 hours, 8 hours, 12 hours or any other time period needed to kill 99.9% of the bacteria, viruses, mold or other microorganism on the surfaces of the goods.

In some embodiments, the environment within the enclosure 1 can be altered to optimize the sanitizing properties of the ozone and/or decrease the potential for ozone to escape and leak to the surrounding environment. For example, the relative humidity, temperature, or ozone concentration can be changed within the sealed enclosure.

In one embodiment, a humidification device 5 is used to increase the relative humidity inside the enclosure 1 for at least a portion of the time that the ozone is circulating to sanitize the goods. In other embodiments, a dehumidification device 12 is used to decrease the relative humidity inside the enclosure for at least a portion of the period of time that the ozone is circulating to sanitize the goods.

Once, the ozone has been circulated for an amount of time sufficient for sanitizing the goods contained within the enclosure, the ozone has to be removed so that the ozone concentration reaches a prescribed safe concentration level. While removing/destroying the ozone, the seal of the enclosure should be maintained until the ozone concentration reaches a prescribed safe concentration level. To remove the ozone to the prescribed safe concentration level, the ozone destructor 3 is engaged to destroy the ozone thermally or catalytically.

After the ozone has been removed to a prescribed safe concentration level, the sealed enclosure can be opened and the sanitized hard and soft goods can be removed.

In some embodiments, the goods can be dried by activating the fresh warm air heater 4 to introduce fresh warm air into the enclosure. In some other embodiments, the goods can be dried by circulating dry air (i.e. air with low humidity) in the enclosure.

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A method of sanitizing hard and soft goods comprising the steps of:
(a) placing the hard and soft goods within an a sealable enclosure;
(b) sealing the enclosure;
(c) producing ozone containing air within the sealed enclosure;
(d) circulating the ozone containing air within the sealed enclosure;
(e) adjusting the environment within the enclosure;
(f) removing the ozone to a prescribed safe concentration level.
(g) maintaining the seal of the enclosure until the ozone concentration reaches a prescribed safe concentration level.

2. The method of claim 1 wherein the prescribed safe concentration level is equal to or less than 1 part per million (ppm) within the enclosure.

3. The method of claim 1 further comprising the steps of opening the sealed enclosure and removing the hard and soft goods.

4. The method of claim 1 wherein the duration of the step of circulating the ozone containing air to sanitize the goods ranges from about 10 minutes to about 30 minutes.

5. The method of claim 1 wherein the step of adjusting the environment in the sealed enclosure comprises changing and maintaining the relative humidity inside the sealed enclosure to greater than 60% for at least a portion of the time.

6. The method of claim 1 wherein the step of adjusting the environment in the sealed enclosure comprises changing and maintaining the relative humidity inside the sealed enclosure to greater than 80% for at least a portion of the time.

7. The method of claim 1 wherein the step of adjusting the environment in the sealed enclosure comprising changing the temperature inside the sealed enclosure.

8. The method of claim 1 wherein leakage of the ozone from the sealed enclosure is minimized by reducing the pressure inside the sealed enclosure to less than 1 atmosphere.

9. The method of claim 1 further comprises using a catalyst to remove the ozone to the prescribed safe concentration level.

10. The method of claim 1 further comprising the step of drying the goods after the ozone is removed.

11. The method of claim 10 wherein the drying step comprises introducing fresh warm air into the sealed enclosure after removing the ozone.

12. The method of claim 10 wherein the drying step comprises circulating air at a low humidity in the sealed enclosure after removing the ozone.

13. An enclosure for sanitizing hard and soft goods comprising:
a sealable enclosure including
an open side;
a sealing door attached to the open side for sealing the sealable enclosure;
a locking mechanism;
an air circulation device for circulating air containing ozone inside the enclosure;
an ozone generator for producing ozone inside the enclosure to create air containing ozone;
an ozone destructor; and
a humidification device to adjust the relative humidity inside the sealed enclosure.

14. The enclosure for sanitizing hard and soft goods according to claim 13 wherein the ozone destructor is a catalyst.

15. The enclosure for sanitizing hard and soft goods according to claim 13 wherein the relative humidity inside the sealed enclosure is greater than 80% for at least a portion of the time the air containing ozone is circulated.

16. The enclosure for sanitizing hard and soft goods according to claim 13 wherein the relative humidity inside the sealed enclosure is greater than 60% for at least a portion of the time the air containing ozone is circulated.

17. The enclosure for sanitizing hard and soft goods according to claim 13 further comprising
an ozone detector.

18. The enclosure for sanitizing hard and soft goods according to claim 17, wherein the ozone detector is disposed within the sealed enclosure.

19. The enclosure for sanitizing hard and soft goods according to claim 17, further comprising another ozone detector wherein the other ozone detector is disposed on an exterior surface of the sealable enclosure for detecting ozone leakage from the enclosure to the external environment above a prescribed safe level.

20. The enclosure for sanitizing hard and soft goods according to claim 13 further comprising a dehumidification device.

21. The enclosure for sanitizing hard and soft goods according to claim 13 further comprising
a cooling unit to decrease the temperature inside the enclosure.

22. The enclosure for sanitizing hard and soft goods according to claim 13, wherein the ozone generator and ozone destructor are disposed within the sealable enclosure.

23. The enclosure for sanitizing hard and soft goods according to claim 13, wherein the ozone generator and ozone destructor are disposed exterior to and fluidly communicate with the sealable enclosure.

24. The enclosure for sanitizing hard and soft goods according to claim 13 further comprising
a fresh air heater.

25. The enclosure for sanitizing hard and soft goods according to claim 13 further comprising
a vacuum pump or blower.
